SEQUENCE LISTING (2 sequences)

SEQ ID NO: 1 - protein, 147 aa, Homo sapiens
APTSSSTKKTQLQLEHLLLDLQNILNGINNYKNPKLTRMLTFKFYMPKKATELKHLQCLEEELKPLEEVLNLAQSKNFHLRPRDLISNINVIVLELKGSETTFNCEYADETATIVEFLNRWITFSQSIISTLTGGSSCRGDKGPDCA

SEQ ID NO: 2 - protein, 147 aa, Homo sapiens
CRGDKGPDCAGGSSAPTSSSTKKTQLQLEHLLLDLQNILNGINNYKNPKLTRMLTFKFYMPKKATELKHLQCLEEELKPLEEVLNLAQSKNFHLRPRDLISNINVIVLELKGSETTFNCEYADETATIVEFLNRWITFSQSIISTLT

(19) United States
(12) Patent Application Publication
(10) Pub. No.: US 2020/0282013 A1
(43) Pub. Date: Sep. 10, 2020

(54) LOW-DOSE CYTOKINE CO-ADMINISTERED WITH IRGD FOR TREATING CANCER

(71) Applicant: DrugCendR, Inc., La Jolla, CA (US)

(72) Inventors: Harri Jarvelainen, Solana Beach, CA (US); Erkki Ruoslahti, Rancho Santa Fe, CA (US)

(21) Appl. No.: 16/812,107

(22) Filed: Mar. 6, 2020

Related U.S. Application Data
(60) Provisional application No. 62/815,917, filed on Mar. 8, 2019.

(51) Int. Cl. A61K 38/12; A61K 38/20; A61P 35/00

(52) U.S. Cl. CPC A61K 38/12; A61P 35/00; A61K 38/2013

(57) ABSTRACT

Methods and compositions comprising iRGD co-administered with cytokines for treating cancer are provided.

### RELATED APPLICATION

The present application claims priority to U.S. provisional patent application No. 62/815,917 filed Mar. 8, 2019, which is incorporated herein by reference in its entirety.

### FIELD OF INVENTION

The invention is related to the co-administration of iRGD (internalized-arginylglycylaspartic acid cyclic peptide; also known as CEND-1) with a cytokine for the treatment of cancer.

### BACKGROUND

### Related Art

Interleukin-2 is a naturally occurring cytokine first discovered in 1976. It is primarily produced by activated T lymphocytes (CD4+ and CD8+ T cells) in response to stimulation. IL-2, and other members of the 4α-helix bundle family of cytokines sharing the same receptors, including IL-4, IL-7, IL-9, IL-15, IL-21, play pivotal roles in the control of the life and death of lymphocytes and activation of adaptive immune responses.

Aldesleukin is a recombinant human IL-2 that became the first FDA-approved cancer immunotherapy in 1992. The approved indications are metastatic renal cell carcinoma and metastatic melanoma. The high-dose IL-2 therapy is mostly used a last-resort treatment for patients with no other therapy options. The efficacy of IL-2 is demonstrated by durable responses in up to 10% of patients. Toxic adverse effects, which include life-threatening and sometimes fatal vascular leak syndrome (VLS), and the dosing regimen of three times per day over eight days necessitated by its short half-life, have limited the clinical usefulness of Aldesleukin. It can only be given to the healthiest patients and only in intensive-care units at specialized medical centers.

Interleukin-2 acts on cell surface receptors on immune cells and stimulates a cytokine cascade involving various types of related interleukins (e.g. IL-1, IL-6, IL-15), interferons (IFN-gamma) and tumor necrosis factor (TNF alpha and beta). IL-2 has a dual role as an immunomodulator, as its pharmacological effect depends on the level of exposure/local concentration at the target tissue. Unfortunately, low concentrations, which would be non-toxic, stimulate regulatory T (Treg) cells, an effect undesirable in the context of cancer immunotherapy. Accordingly, attempts to test low-dose IL-2 therapy for cancer have been disappointing, presumably in part, due to the expansion of Treg cells (Waldmann, 2015, Cancer Immunol Res. 3: 219-227). In contrast, the anti-tumor activity of IL-2 is believed to result from activation of cytotoxic CD8+ T cells, which only occurs at high intratumor concentrations of IL-2. Unfortunately, the high systemic dose levels required to achieve and maintain these therapeutically beneficial IL-2 levels within the tumor cause severe systemic toxicities.

### SUMMARY

Provided herein is a method for treating cancer in a patient in need thereof, wherein the method comprises administering, to a patient in need thereof, iRGD (CEND-1); and a low cumulative dose of a cytokine. In particular embodiments, the cancer can be selected from the group consisting of: Bladder Cancer, Breast Cancer, Cervical Cancer, Colon & Rectal cancer, Endometrial Cancer, Kidney Cancer, Lip & Oral Cancer, Liver Cancer (e.g., renal cell carcinoma), Melanoma, Mesothelioma, Non-Small Cell Lung Cancer, Nonmelanoma Skin Cancer, Oral Cancer, Ovarian Cancer, Pancreatic Cancer, Prostate Cancer, Sarcoma, Small Cell Lung Cancer, and Thyroid Cancer. In particular embodiments, the low cumulative dose is selected from the group consisting of; about 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 120-fold, 140-fold, 160-fold, 180-fold, 190-fold, 200-fold, 300-fold, 400-fold, 500-fold, 600-fold, 700-fold, 800-fold, 900-fold and 1,000-fold lower than the amount of dose that is known in the art to be the starting dose for either a respective human patient or animal model. In yet other embodiments, the cytokine is Aldesleukin or IL-2.

Also provided herein is a method for treating, inhibiting, or reducing the volume of a tumor in a subject or patient in need thereof, wherein the method comprises administering iRGD (CEND-1); and a cytokine. In one embodiment, the cytokine can be selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine is selected from the group consisting of: IL-2, Aldesleukin, IL-4, IL-6, IL-7, IL-10, IL-12, IL-13, IL-15. In yet another embodiment, the cytokine is selected from IL-2 or Aldesleukin.

In a particular embodiment, the iRGD and cytokine are co-administered to the subject or patient. In another embodiment, the method further comprises the steps of: (1) intravenous injection of iRGD; and (2) administering intravenous IL-2. In a particular embodiment, the cytokine is administered at a low cumulative dose.

Also provided herein, are compositions comprising iRGD (CEND-1); and a cytokine. In one embodiment, the cytokine is selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine can be selected from the group consisting of: IL-2, Aldesleukin, IL-4, IL-6, IL-7, IL-10, IL-12, IL-13, IL-15. In a particular embodiment, the cytokine can be selected from IL-2 or Aldesleukin. In yet another embodiment, the iRGD and cytokine are in the form of a recombinant fusion protein or a covalently linked chemical conjugate.

Also provided are kits comprising iRGD (CEND-1); and a cytokine. In one embodiment, the cytokine can be selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-13, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine can be selected from the group consisting of: IL-2, Aldesleukin, IL-4, IL-6, IL-7, IL-10, IL-12, IL-13, IL-15. In a particular embodiment, the cytokine is selected from IL-2 or Aldesleukin.

Other features and advantages of the present invention will become more readily apparent to those of ordinary skill in the art after reviewing the following detailed description and accompanying drawings.

### BRIEF DESCRIPTION OF THE DRAWINGS

FIG. 1 shows the percentages of total T cells (CD3) in the tumor.

FIG. 2 shows the percentage of CD4 T cells in the tumor.

FIG. 3 shows the percentage of Treg of the total T cells.

FIG. 4 shows the ratios of CD4 Teff/Treg in 4T1 tumor.

FIG. 5 shows the percentages of CD4 T cells in the tumor.

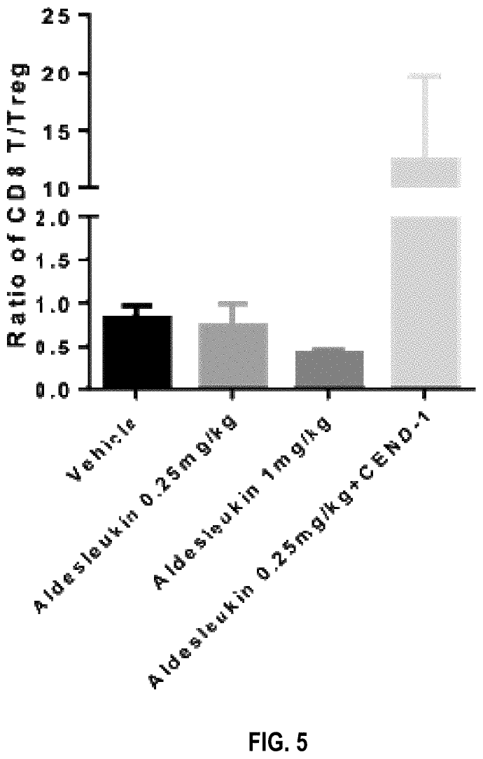

FIG. 6 shows the immune cell profiling tree as depicted in Table 3.

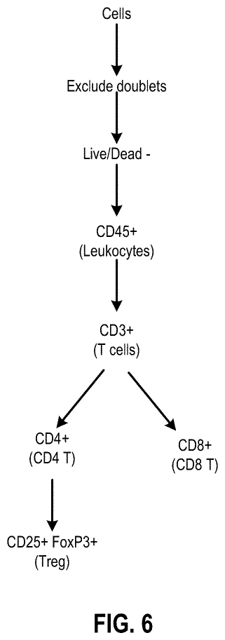

### DETAILED DESCRIPTION

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

Provided herein is a method for treating cancer in a patient in need thereof, wherein the method comprises administering, to a patient in need thereof, iRGD (CEND-1); and a low cumulative dose of a cytokine In one embodiment, the cytokine can be selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine is selected from the group consisting of: IL-2, Aldesleukin, IL-4, IL-6, IL-7, IL-10, IL-12, IL-13, IL-15. In yet another embodiment, the cytokine is selected from IL-2 or Aldesleukin.

In accordance with the present invention, it has been found that the combination treatment of iRGD with low cumulative doses of a cytokine (e.g, IL-2, or the like) is capable of favorably altering the pharmacology of IL-2, leading to changes in tumor immune microenvironment such that immunosuppressive Treg cells are reduced with a concomitant increase in effector T-cell populations. The tumor-selective Interleukin pharmacology benefit obtained with iRGD is contemplated herein to provide new options for the use of the well-validated IL-2 and other related cytokines in solid tumor cancer patients, including a strategy to overcome primary resistance to PD-1 blockade.

Different types of solid tumors, and solid tumor cancers, are contemplated for treatment herein by the invention methods and are generally named for the type of cells that form them. Examples of solid tumors are sarcomas, carcinomas, and lymphomas. Accordingly solid tumor cancers for treatment by the invention methods include, among others, Bladder Cancer, Breast Cancer, Cervical Cancer, Colon & Rectal cancer, Endometrial Cancer, Kidney Cancer, Lip & Oral Cancer, Liver Cancer (e.g., renal cell carcinoma), Melanoma, Mesothelioma, Non-Small Cell Lung Cancer, Nonmelanoma Skin Cancer, Oral Cancer, Ovarian Cancer, Pancreatic Cancer, Prostate Cancer, Sarcoma, Small Cell Lung Cancer, Thyroid Cancer.

The iRGD molecular mimicry technology has been found to turn a normally difficult-to-access tumor microenvironment into a drug conduit, allowing efficient access of anti-cancer agents deep into the tumor (Ruoslahti, 2017, Adv Drug Deliv Rev. 110-111:3-12). In accordance with the present invention, co-administered anti-cancer agents (e.g., cytokines, such as IL-2, and the like) become more tumor-targeted, with a better efficacy and/or reduced systemic side-effects. The effect of iRGD co-administration on IL-2 has been found to achieve enough of a reduction of the dose to circumvent the most serious toxicities. In one embodiment, it has been unexpectedly found that favorable changes in tumor immune profile can be achieved with low and non-toxic doses of IL-2, that without iRGD are pharmacologically inactive or immunosuppressive. Particularly remarkable was the reversal of the Treg cell-promoting activity of low-dose IL-2 into an anti-Treg activity. Together with the increase of T effector cell levels that was obtained, the IL-2/iRGD combination converted this toxic cytokine into an active and non-toxic compound.

In a particular embodiment, the iRGD and cytokine are co-administered to the subject or patient. As used herein “co-administration” refers to the substantially simultaneous administration of the iRGD and respective cytokine, such that the iRGD functions to activate the ‘CendR’ transcytosis and trans-tissue transport pathway, and thereby increase tumor penetration and accumulation of various types of co-administered drugs. In another embodiment, the method further comprises the steps of: (1) intravenous injection of iRGD; and (2) administering intravenous IL-2. In a particular embodiment, the cytokine is administered at a low cumulative dose.

In accordance with the present invention, it has been found that co-administration of a cytokine (e.g., IL-2) with iRGD peptide converts a low and inefficient, but essentially non-toxic dose of IL-2 into an efficient inducer of lymphocyte recruitment into tumors, and that the profile of the lymphocytes is conducive to anti-tumor immunity. Remarkably, these changes were observed at an IL-2 dose that is several times lower than the dose levels commonly reported to be efficient in other comparable mouse studies. As an example, Charych et al. (2016) used a cumulative IL-2 dose of 35 mg/kg (3 mg/kg b.i.d. for 5 days); whereas in one embodiment of the present invention methods, the lowest cumulative dose found to be effective is 1.25 mg/kg (0.25 mg/kg once daily for 5 days); which corresponds to a 28-fold lower cumulative dose than the dose levels commonly reported or known in the art to be effective. In accordance with the present invention, the IL-2 low cumulative dose levels were also devoid of any adverse clinical signs or changes in clinical pathology (clinical chemistry and hematology) parameters.

As set forth herein, a surprising feature of the invention methods and compositions is the large factor by which we can reduce the IL-2 dose. Table 1 below shows that the IL-2 low dose used (660,000 IU/day) with co-administration of iRGD is about 190-fold lower than the standard IL-2 dose 126,000,000 IU/day) used in cancer therapy. In other embodiments, when iRGD is co-administered with other cancer drugs or cytokines the difference is typically a 3-4-fold lower cumulative dose.

TABLE 1

| Comparison | Low Dose IL-2 | High Dose IL-2 | iRGD + IL-2 |
|---|---|---|---|
| Use | HSCT to | RCC and | Concentrate |
| | increase | Melanoma to | systemic |
| | Tregs and | amplify CD8+ | low dose IL-2 |
| | decrease | cytotoxic | in tumor to |
| | GVHD | T-cells | achieve high |
| | | and induce | dose IL-2. |
| | | remission | Decrease Tregs |
| | | | and amplify |
| | | | CD8+ T-cells. |
| Dosing used | 1,000,000 | 126,000,000 | 660,000 |
| | IU/day | IU/day | IU/day |
| % vs iRGD + IL-2 | 150% more | 19,090% more | — |
| Time Period | 12 weeks | Days 1-5 and 15- | 5 days, 5 doses |
| | | 19, TID, max28 | |
| | | doses | |

Accordingly, in one embodiment, a “low dose” or “low cumulative dose” as used refers to a cumulative dose of cytokine (e.g., IL-2) that is several times lower than the dose levels commonly reported or known in the art to be effective, although they may produce side-effects, in treating the respective solid tumor or cancer; or in a comparable animal model. For example, High-dose interleukin-2 (HD IL-2) was approved for treatment of metastatic renal cell carcinoma (mRCC) in 1992 and for metastatic melanoma (mM) in 1998, in an era predating targeted therapies and immune checkpoint inhibitors (see, Alva et al., Cancer Immunol Immunother. 2016; 65(12): 1533-1544). Alva et al. indicate that physicians managed and treated patients per each institution's standard of care and their own clinical judgment. High-dose IL-2 (Proleukin®) was administered as an intravenous bolus every 8 h at a dose of 600,000 IU/kg or 720,000 IU/kg as tolerated, with up to 14 consecutive doses over 5 days (1 cycle of therapy). Thus, the 5-day cumulative doses equate to 8,400,000 IU/kg or 10,080,000 IU/kg respectively for 1 cycle of therapy. As with these known High-dose methods of treating cancer, a cycle of therapy of the invention low-dose method can be repeated as needed, such after a rest period of approximately 9-days, or the like. As another example, from Table 1 is indicated that the standard (“high dose”) of IL-2 for treating RCC and Melanoma is 126,000,000 IU/day.

In one particular embodiment, about 1/35th the amount of dose that is known in the art to be the starting dose (e.g., High dose) for either a respective human patient or animal model, is employed. In other embodiments, a low cumulative dose can be selected from the group of ranges consisting of: about 1/1000th up to about 1/500th, 1/1000^{th} up to about 1/190^{th}, 1/1000th up to about 1/100th, 1/1000th up to about 1/75th, 1/1000th up to about 1/50th, 1/1000th up to about 1/35th, 1/1000th up to about 1/25th, 1/1000th up to about 1/10th, 1/1000th up to about ⅕th, 1/1000th up to about ⅓rd, and 1/1000th up to about ½th the amount of dose that is known in the art to be the starting dose for either a respective human patient or animal model. In other embodiments, a low cumulative dose can be selected from the group consisting of: about 1/1000th, 1/500th, 1/190^{th}, 1/120^{th}, 1/100th, 1/75th, 1/50th, 1/35th, 1/25th, 1/10th, ⅕th, ⅓rd, and ½th the amount of dose that is known in the art to be the starting dose for either a respective human patient or animal model.

In another embodiment, a “low dose” or “low cumulative dose” can be from about: 2-fold to about 1000-fold; 3-fold to about 500-fold, 4-fold to about 300-fold, 5-fold to about 200-fold, 10-fold to about 190-fold, 10-fold to about 150-fold, 10-fold to about 125-fold, and 10-fold to about 100-fold lower than the amount of dose that is known in the art (e.g. such as on an FDA approved drug label, and the like) to be the starting dose (e.g., High dose) for either a respective human patient or animal model. In yet another embodiment, a “low dose” or “low cumulative dose” can be selected from the group consisting of; about 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 120-fold, 140-fold, 160-fold, 180-fold, 190-fold, 200-fold, 300-fold, 400-fold, 500-fold, 600-fold, 700-fold, 800-fold, 900-fold and 1,000-fold lower than the amount of dose that is known in the art (e.g. such as on an FDA approved drug label, and the like) to be the starting dose for either a respective human patient or animal model.

In another embodiment, a “low dose” or “low cumulative dose” can be from about 1 ng/Kg up to about 1 mg/kg; 1 ng/Kg up to about 0.9 mg/Kg, 1 ng/Kg up to about 0.8 mg/Kg, 1 ng/Kg up to about 0.7 mg/Kg, 1 ng/Kg up to about 0.6 mg/Kg, 1 ng/Kg up to about 0.5 mg/Kg, 1 ng/Kg up to about 0.4 mg/Kg, 1 ng/Kg up to about 0.3 mg/Kg, 1 ng/Kg up to about 0.2 mg/Kg, 1 ng/Kg up to about 0.1 mg/Kg. In other embodiments, a low cumulative dose can be selected from the group consisting of: about 1 ng/Kg up to about 10 ug/kg, about 100 ng/Kg up to about 5 ug/kg, about 500 ng/Kg up to about 3 ug/kg, about 750 ng/Kg up to about 2 ug/kg, about 1 ug/Kg up to about 1.5 ug/kg. In other embodiments, a low cumulative dose can be selected from the group consisting of: about 0.1 ng/Kg up to about 10 ug/kg, about 0.1 ng/Kg up to about 5 ug/kg, about 0.1 ng/Kg up to about 3 ug/kg, about 0.1 ng/Kg up to about 2 ug/kg, about 0.1 ng/Kg up to about 1.5 ug/kg, and about 0.1 ng/Kg up to about 0.1 ug/kg, and the like. In yet other embodiments, a low cumulative dose can be selected from the group consisting of: about 0.01 ng/Kg up to about 100 ng/kg, about 0.01 ng/Kg up to about 90 ng/kg, about 0.01 ng/Kg up to about 80 ng/kg, about 0.01 ng/Kg up to about 70 ng/kg, about 0.01 ng/Kg up to about 60 ng/kg, 0.01 ng/Kg up to about 50 ng/kg, about 0.01 ng/Kg up to about 40 ng/kg, about 0.01 ng/Kg up to about 30 ng/kg, about 0.01 ng/Kg up to about 20 ng/kg and about 0.01 ng/Kg up to about 10 ng/kg, and the like.

Several approaches taken to improve the safety profile of Aldesleukin have been reported. However, these approaches involve modification of the structure of IL-2, generally aiming at changing the receptor binding profile in order to mitigate toxicities. Although these modified IL-2 compounds have lower toxicity than Aldesleukin, the efficacy is also reduced. Accordingly, none of the previous low-dose IL-2 approaches have proven effective in the clinic. A further drawback of non-natural versions of IL-2 is a greater risk for immunogenicity.

In accordance with the present invention, co-administration with iRGD altered the pharmacology of low-dose IL-2 by tipping the balance in favor of CD8+ T-cells over Treg cells, a change that favors anti-tumor immunity. These changes in the immunostimulatory profile were obtained without any changes in the structure of the recombinant protein that could negatively affect the efficacy, safety or immunogenicity. In accordance with the present invention, a new treatment method is provided in which cytokines, such as IL-2, are used in cancer immunotherapy at low cumulative doses when combined with iRGD, achieving efficacy while avoiding the toxicity caused by the fulminant systemic immune activation elicited by cytokines at the currently used doses.

In other embodiments, the low cumulative doses of cytokine (e.g., IL-2, and the like) contemplated for use herein with iRGD, in human cancer patients, are selected from the group consisting of no greater than: 1 mg/kg, 0.9 mg/kg, 0.8 mg/kg, 0.75 mg/kg, 0.7 mg/kg, 0.6 mg/kg, 0.5 mg/kg, 0.4 mg/kg, 0.3 mg/kg, 0.25 mg/kg, 0.2 mg/kg and 0.1 mg/kg. In yet other embodiments, the low cumulative doses of cytokine (e.g., IL-2, and the like) contemplated for use herein with iRGD, in human cancer patients, are selected from the group consisting of no greater than: 100 ng/kg, 90 ng/kg, 80 ng/kg, 70 ng/kg, 60 ng/kg, 50 ng/kg, 40 ng/kg, 30 ng/kg, 20 ng/kg, 17.5 ng/kg, 15 ng/kg, 12.5 ng/kg, 10 ng/kg, 9 ng/kg, 8 ng/kg, 7.5 ng/kg, 7 ng/kg, 6 ng/kg, 5 ng/kg, 4 ng/kg, 3 ng/kg, 2.5 ng/kg, 2 ng/kg, 1 ng/kg, 0.9 ng/kg, 0.8 ng/kg, 0.7 ng/kg, 0.6 ng/kg, 0.5 ng/kg, 0.4 ng/kg, 0.3 ng/kg, 0.2 ng/kg and 0.1 ng/kg.

Also provided herein, are compositions comprising iRGD (CEND-1); and a cytokine. In one embodiment, the cytokine is selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine can be selected from the group consisting of: IL-2, Aldesleukin, IL-4, IL-6, IL-7, IL-10, IL-12, IL-13, IL-15. In a particular embodiment, the cytokine can be selected from IL-2 or Aldesleukin. In yet another embodiment, the iRGD and cytokine compositions are in the form of a recombinant fusion protein or a covalently linked chemical conjugate.

In addition to co-administration of the cytokine, e.g., IL-2, with iRGD, it Is also contemplated that fusion proteins or conjugates of the cytokine e.g. IL-2/iRGD, will result in even more efficient and targeted tumor targeting. For example, the following recombinant fusion of IL-2/iRGD is contemplated for use herein, where amino acids 1-133 correspond to secreted IL-2, with the signal peptide; and amino acids 138-147 correspond to iRGD separated by a 4 amino acid linker domain (underlined):

(SEQ ID NO: 1)
APTSSSTKKTQLQLEHLLLDLQNILNGINNYKNPKLTRMLTFKFYMPK
KATELKHLQCLEEELKPLEEVLNLAQSKNFHLRPRDLISNINVIVLEL
KGSETTFNCEYADETATIVEFLNRWITFSQSIISTLTGGSSCRGDKGP
DCA

Also contemplated for use herein is iRGD sequence at the amino terminus of the of the fusion protein separated from IL-2 by the same 4 amino acid linker domain (underlined) as follows:

(SEQ ID NO: 2)
CRGDKGPDCAGGSSAPTSSSTKKTQLQLEHLLLDLQNILNGINNYKNP
KLTRMLTFKFYMPKKATELKHLQCLEEELKPLEEVLNLAQSKNFHLRP
RDLISNINVIVLELKGSETTFNCEYADETATIVEFLNRWITFSQSIIS
TLT

Numerous other amino acid or polypeptide linker domains well-known in the art are contemplated here for use in the cytokine (e.g., IL-2)/iRGD recombinant fusion proteins. In other embodiments, the fusion proteins of the invention can employ one or more “linker domains,” such as polypeptide linkers. As used herein, the term “linker domain” refers to a sequence which connects two or more domains in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect a cytokine domain to the iRGD domain. Such polypeptide linkers can provide flexibility to the fusion proteins. In certain embodiments the polypeptide linker can be used to connect (e.g., genetically fuse) one or more cytokine domains and/or one or more iRGD domains. A fusion protein of the invention may comprise more than one linker domain or peptide linker.

As used herein, the term “gly-ser polypeptide linker” refers to a peptide that consists of glycine and serine residues. Another exemplary gly/ser polypeptide linker comprises the amino acid sequence Ser(Gly4Ser)n, where n is 1-20. For example, in one embodiment, n=3, i.e., Ser(Gly4Ser)3. In another embodiment, n=4, i.e., Ser(Gly4Ser)4, and the like.

In addition to recombinant fusion proteins, chemical conjugates of the cytokine (e.g., IL-2)/iRGD polypeptides are contemplated herein for use in the invention methods. These cytokine/iRGD conjugates can be represented by the following formula:

C-L-iRGD;

where C is a cytokine (e.g., IL-2, L is a chemical linker and iRGD is internalized-arginylglycylaspartic acid cyclic peptide or CEND-1 (see U.S. Pat. Nos. 8,367,621; 9,115,170; and the like; each of which are incorporated by reference in their entirety for all purposes). In one embodiment, the cytokine/iRGD conjugate provided herein is IL-2 (or Aldesleukin)-L-iRGD.

Exemplary chemical linker functional groups for use herein are well-known in the art, and include amino (—NRH), carboxylic acid (—C(O)OH) and derivatives, sulfonic acid (—S(O)2-OH) and derivatives, carbonate (—O—C(O)—O—) and derivatives, hydroxyl (—OH), aldehyde (—CHO), ketone (—CRO), isocyanate (—NCO), isothiocyanate (—NCS), haloacetyl, alkyl halides, maleimide, acryloyl, arylating agents like aryl fluorides, disulfides like pyridyl disulfide, vinyl sulfone, vinyl ketone, diazoalkanes, diazoacetyl compounds, epoxide, oxirane, and/or aziridine. Nonlimiting examples of R include H, linear, branched or cyclical alkyl groups which may contain further functional groups or hetero atoms or aryl groups.

As used herein, a “chemical linker” is a molecule that serves to join other atoms, molecules, or functional groups together via covalent or non-covalent interactions. Exemplary monomeric, polymeric and other suitable linkers useful herein for conjugating biological molecules are set forth in U.S. Pat. Nos. 8,546,309; 8,461,117; 8,399,403; 10, 550,190; 10,557,644; 10,519,265; each of which are incorporated by reference in their entirety for all purposes.

In view of the data provided herein in accordance with the present invention, testing of IL-2/iRGD in combination with other immunotherapies is also contemplated herein. For example, IL-2 has shown promise when used in combination with checkpoint inhibitor antibodies such as PD-1 inhibitors. There is currently a collection of ongoing studies using Aldesleukin across 40 participating sites (PROleukin Observational Study to Evaluate the Treatment Patterns and Clinical Response in Malignancy; NCT01415167). Accordingly, the present invention methods are contemplated herein to provide a therapy-enhancing activity of Aldesleukin when combined with checkpoint inhibitors (e.g. anti-CTLA-4; ipilimumab, Yervoy® and the PD-1 inhibitors (pembrolizumab and nivolumab). Thus, in particular embodiments, the invention methods further comprise administration of a low cumulative dose of cytokine (e.g., IL-2) and iRGD, in combination with the administration of a checkpoint inhibitor selected from the group consisting of: ipilimumab (Yervoy®), pembrolizumab (Keytruda®), nivolumab (Opdivo®), atezolizumab (Tecentriq®), avelumab (Bavencio®), durvalumab (Imfinzi®), and cemiplimab (Libtayo®).

That IL-2 is clinically validated anti-cancer drug, and that iRGD is undergoing clinical testing in cancer patients, will greatly facilitate the introduction of the IL-2/iRGD combination into the clinic.

Also provided are kits comprising iRGD (CEND-1); and a cytokine. In one embodiment, the cytokine can be selected from the group consisting of: IL-1-like, IL-1α, IL-1B, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP. In another embodiment, the cytokine can be selected from the group consisting of: IL-2, Aldesleukin, IL-4, IL-6, IL-7, IL-10, IL-12, IL-13, IL-15. In a particular embodiment, the cytokine is selected from IL-2 or Aldesleukin.

Also provided are kits for practicing the subject methods. While the subject kits may vary greatly in regards to the components included, typically, the kits at least include at least one cytokine (e.g., IL-2) and an iRGD in a suitable form. The subject kits may also include one or more other pharmacological agents. The dosage amount of the one or more cytokine and iRGD and/or other pharmacological agents provided in a kit may be sufficient for a single application or for multiple applications. Accordingly, in certain embodiments of the subject kits a single dosage amount of a cytokine (e.g., IL-2), iRGD and/or a single dosage of at least one another, different pharmacological agent is present.

In certain other embodiments, multiple dosage amounts of a cytokine (e.g., IL-2), iRGD and/or one other pharmacological agent may be present in a kit. In those embodiments having multiple dosage amounts of, e.g., at least one such cytokine (e.g., IL-2) and/or iRGD, may be packaged in a single container, e.g., a single tube, bottle, vial, and the like, or one or more dosage amounts may be individually packaged such that certain kits may have more than one container of a a cytokine (e.g., IL-2) and/or iRGD.

Suitable means for delivering one or more a cytokine (e.g., IL-2), iRGD and/or other pharmacological agents to a subject may also be provided in a subject kit. The particular delivery means provided in a kit is dictated by the particular a cytokine (e.g., IL-2), iRGD and/or pharmacological agent employed, as describe above, e.g., the particular form of the a cytokine (e.g., IL-2), iRGD and/or other agent such as whether the a cytokine (e.g., IL-2), iRGD and/or other pharmacological agent is formulated into preparations in solid, semi-solid, liquid or gaseous forms, such as tablets, capsules, powders, granules, ointments, solutions, suppositories, injections, inhalants and aerosols, and the like, and the particular mode of administration of the agent, e.g., whether oral, buccal, rectal, parenteral, intravaginal, endocervical, intrathecal, intranasal, intravesicular, on the eye, in the ear canal, intraperiactivityal, intradermal, transdermal, intracheal, etc. Accordingly, certain systems may include a suppository applicator, syringe, I.V. bag and tubing, electrode, transdermal patch or film, etc.

The subject kits also include instructions for how to practice the subject methods and in particular how to administer the at least one a cytokine (e.g., IL-2) and/or iRGD provided in the kit to treat a subject for a the respective cancer. The instructions are generally recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

One embodiment of the present invention relates to (among other things) a method of administering iRGD to a patient with a solid tumor, the method comprising the steps of: (1) intravenous injection of iRGD (also known as internalized-arginylglycylaspartic acid cyclic peptide or CEND-1); (2) a low cumulative dose of intravenous IL-2 to activate the patient's immune system without the side effects associated with conventional IL-2 therapy.

Recombinant IL-2 at high doses is an effective immunotherapy treatment for various types of solid tumors but its clinical utility has been limited by serious mechanism-based side-effects. The clinical-stage iRGD peptide specifically targets tumors and, via activation of the ‘CendR’ transcytosis and trans-tissue transport pathway, increases tumor penetration and accumulation of various types of co-administered drugs. In accordance with the present invention, co-administration with iRGD reduces the toxicities arising from IL-2, and other cytokines, in non-target tissues by allowing the use of IL-2 in low, non-toxic, doses; and by selectively increasing the IL-2 delivery into tumors, but not to normal tissues.

Subcutaneous breast tumors were generated in immunocompetent mice with 4T1 mouse breast cancer cells. The tumor-bearing mice were treated with a vehicle control, iRGD, IL-2, or IL-2+iRGD for 5 days. Tumors were enzymatically digested for fluorescence activated cell sorting (FACS) 16 hours after the last dosing. The FACS and IHC were used to detect the percentage of total T cells, CD4 and CD8 T cells, and Treg cells.

Low cumulative doses of IL-2 alone were found to increase the level of Treg cells within the tumor but had no effect in CD4 or CD8 effector T cells, compared to vehicle treatment. Surprisingly, low-cumulative-dose IL-2 co-administered with iRGD had the opposite effect on Treg cells; a significantly lower percentage of Treg cells was observed in the tumors. In contrast, the ratio of CD8/Treg cells was increased by at least 10-fold compared to low dose IL-2 alone. The iRGD combination also gave an increase in CD4 effector T cells. Importantly, no adverse side-effects were observed in these experiments.

### Materials and Methods

Cell Culture: 4T1 tumor cells were maintained in vitro as a monolayer culture in RPMI1640 medium supplemented with 10% heat inactivated fetal bovine serum (FBS), 100 U/ml penicillin and 100 μg/ml streptomycin at 37° C. in an atmosphere of 5% CO2 in air. The tumor cells were routinely subcultured twice weekly by trypsin-EDTA treatment. The cells growing in an exponential growth phase were harvested and counted for tumor inoculation.

Animals and dosing information: A total of 21 female BALB/c mice, 6-8 weeks of age, weighing approximately 18-22 g, were used for the study. Animals were purchased from Shanghai SLAC Laboratory Animal Co., LTD and marked by ear punching prior to the inoculation of 4T1 cancer cells. Each mouse was inoculated subcutaneously at the right flank with the cells (1×105) in 0.1 ml of PBS without anesthesia for tumor development. Daily i.v. dosing with Aldesleukin with or without iRGD (CEND-1) was initiated when the tumor volume exceeded 100 mm3 and the treatment regimen was continued for 5 days (see Table 2 for treatment information).

TABLE 2

| | | | Dose |
|---|---|---|---|
| Group | n | Treatment | (mg/kg) |
| 1 | 6 | Vehicle | — |
| 2 | 5 | Aldesleukin | 0.25 |
| 3 | 3 | Aldesleukin | 1 |
| 4 | 6 | Aldesleukin + iRGD | 0.25 + 4 |

Immune cell profiling—Tumors were harvested 6 days after treatment initiation (24 hours after the last dose). The tumors were mechanically dispersed and enzymatically digested. The FACS antibody panel was designed for determination of the percentage of T, CD4 T, CD8 T and Treg in CD45+ live cells in the tumors (Table 3). Data are presented as a percent of total immune cells isolated.

TABLE 3

| Channel | Marker | Cell subpopulation |
|---|---|---|
| BV421 | Live/Dead | Live/Dead |
| AF700 | CD45 | Leukocyte |
| APC-Cy7 | CD3 | T |
| PerCP-Cy5.5 | CD4 | CD4 T |
| APC | CD8 | CD8 T |
| BV605 | CD25 | Treg |
| PE-Cy7 | FoxP3 | Treg |

Data analysis—Statistical analysis was performed using ANOVA, with Tukey's post-test.

### Results

The low IL-2 doses used in this study were well tolerated and were not associated with any adverse clinical signs, changes in food consumption or weight gain. There were no changes in clinical chemistry or hematology parameters analyzed.

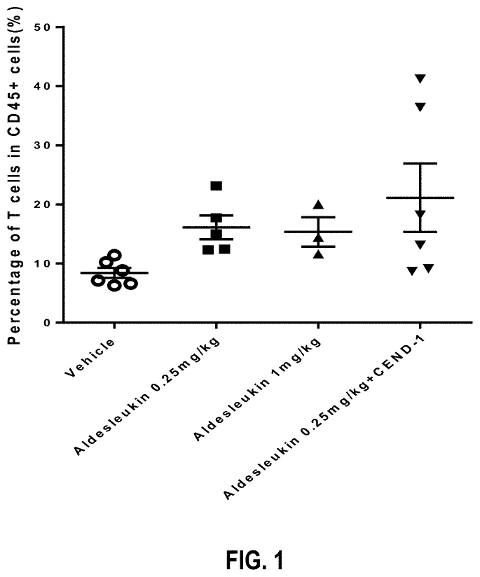

Quantification of CD3+ T cells in tumor mice treated with Aldesleukin at 0.25 mg/kg (with or without iRGD), or 1 mg/kg showed significantly increased percentage of of CD3+ cells in each group compared with vehicle controls (FIG. 1; statistical significance not indicated in the graph).

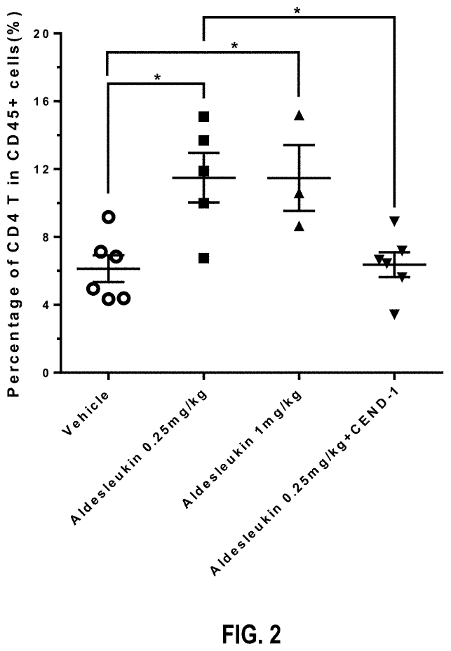

The percentage of CD4 T in the 0.25 mg/kg of Aldesleukin+CEND1 combo group was significantly lower than in the group treated with the same dose of Aldesleukin alone (FIG. 2).

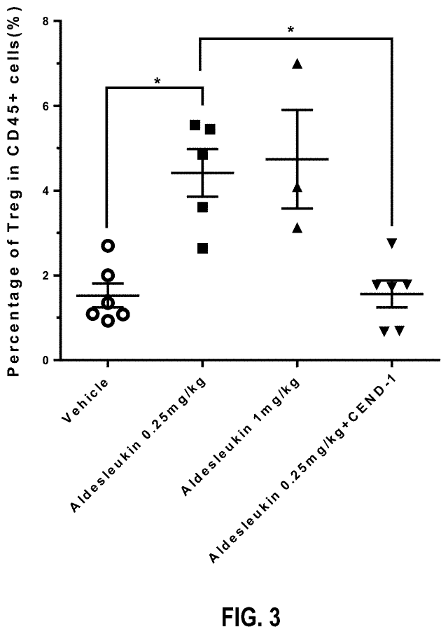

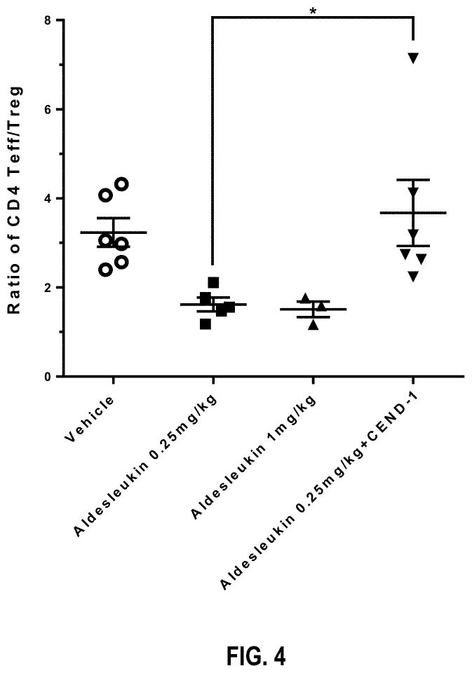

The percentage of Treg cells in the 0.25 mg/kg Aldesleukin (alone) group increased significantly compared with vehicle control, whereas the Aldesleukin+CEND1 combo significantly lowered the Treg cell count relative to the control group (FIG. 3). Moreover, the ratio of CD4 effector T cells (Teff) to Treg cells decreased significantly the 0.25 mg/kg and 1 mg/kg Aldesleukin groups, whereas the CD4 Teff/Treg ratio increased in Aldesleukin+CEND-1 combination group (FIG. 4; CD4 Teff=total CD4 T−Treg). In addition, a tendency toward an increased CD8 T/Treg ratio was observed in the combo group.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

### SEQUENCE LISTING

The sequence listing submitted herewith in the ASCII text file entitled “Sequence_Listing_ST25_127380-001UT1,” created May 1, 2020, with a file size of 2.953 kilobytes, is incorporated herein by reference in its entirety.

What is claimed is:

1. A method for treating, inhibiting, or reducing the volume of a tumor in a subject or patient in need thereof, wherein the method comprises administering iRGD (CEND-1); and a cytokine.

2. The method of claim 1, wherein the cytokine is selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP.

3. The method of claim 1, wherein the cytokine is selected from the group consisting of: IL-2, Aldesleukin, IL-4, IL-6, IL-7, IL-10, IL-12, IL-13, IL-15.

4. The method of claim 1, wherein the cytokine is selected from IL-2 or Aldesleukin.

5. The method of claim 1, wherein the iRGD and cytokine are co-administered to the subject or patient.

6. The method of claim 1, wherein the method further comprises the steps of: (1) intravenous injection of iRGD; and (2) administering intravenous IL-2.

7. The method of claim 1, wherein the cytokine is administered at a low cumulative dose.

8. A composition comprising iRGD (CEND-1); and a cytokine.

9. The composition of claim 8, wherein the cytokine is selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP.

10. The composition of claim 9, wherein the cytokine is selected from the group consisting of: IL-2, Aldesleukin, IL-4, IL-6, IL-7, IL-10, IL-12, IL-13, IL-15.

11. The composition of claim 10, wherein the cytokine is selected from IL-2 or Aldesleukin.

12. The composition of claim 8, wherein the iRGD and cytokine are in the form of a recombinant fusion protein or a covalently linked chemical conjugate.

13. A kit comprising iRGD (CEND-1); and a cytokine.

14. The kit of claim 13, wherein the cytokine is selected from the group consisting of: IL-1-like, IL-1α, IL-1β, IL-1RA, IL-18, IL-2, IL-4, IL-7, IL-9, IL-13, IL-15, L-3, IL-5, GM-CSF, IL-6-like, IL-6, IL-11, G-CSF, IL-12, LIF, OSM, IL-10-like, IL-10, IL-20, IL-14, IL-16, IL-17 IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, Epo, Tpo, Flt-3L, SCF, M-CSF, MSP.

15. The kit of claim 13, wherein the cytokine is selected from the group consisting of: IL-2, Aldesleukin, IL-4, IL-6, IL-7, IL-10, IL-12, IL-13, IL-15.

16. The kit of claim 13, wherein the cytokine is selected from IL-2 or Aldesleukin.

17. A method for treating cancer in a patient in need thereof, wherein the method comprises administering, to a patient in need thereof, iRGD (CEND-1); and a low cumulative dose of a cytokine.

18. The method claim 17, wherein the cancer is selected from the group consisting of: Bladder Cancer, Breast Cancer, Cervical Cancer, Colon & Rectal cancer, Endometrial Cancer, Kidney Cancer, Lip & Oral Cancer, Liver Cancer (e.g., renal cell carcinoma), Melanoma, Mesothelioma, Non-Small Cell Lung Cancer, Nonmelanoma Skin Cancer, Oral Cancer, Ovarian Cancer, Pancreatic Cancer, Prostate Cancer, Sarcoma, Small Cell Lung Cancer, and Thyroid Cancer.

19. The method of claim 17, wherein the low cumulative dose is selected from the group consisting of; about 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 120-fold, 140-fold, 160-fold, 180-fold, 190-fold, 200-fold, 300-fold, 400-fold, 500-fold, 600-fold, 700-fold, 800-fold, 900-fold and 1,000-fold lower than the amount of dose that is known in the art to be the starting dose for either a respective human patient or animal model.

20. The method of claim 17, wherein the cytokine is Aldesleukin or IL-2
